(19) United States
(12) Patent Application Publication
(10) Pub. No.: US 2026/0203896 A1
(43) Pub. Date: Jul. 16, 2026

(54) METHODS AND SYSTEMS FOR IDENTIFYING REGIONS OF INTEREST FROM THREE-DIMENSIONAL MEDICAL IMAGE DATA

(71) Applicant: University of Washington, Seattle, WA (US)

(72) Inventors: Jonathan Teng-Chieh LIU, Seattle, WA (US); Lindsey A. ERION BARNER, Seattle, WA (US); Gan GAO

(21) Appl. No.: 19/135,796

(22) PCT Filed: Jan. 25, 2024
(86) PCT No.: PCT/US2024/012982

§ 371 Date: Jun. 4, 2025

Related U.S. Application Data
(60) Provisional application No. 63/481,761, filed on Jan. 26, 2023.

(51) Int. Cl. G06T 7/00; G06V 10/25; G06V 10/764; G06V 10/82; G16H 30/40

(52) U.S. Cl. CPC G06T 7/0012; G06V 10/25; G06V 10/764; G06V 10/82; G16H 30/40; G06T 2200/04; G06T 2207/10056; G06T 2207/20076; G06T 2207/20081; G06T 2207/20084; G06T 2207/30081; G06T 2207/30092; G06V 2201/03

(57) ABSTRACT

Embodiments of the present disclosure provide methods and systems for identifying regions of interest from three-dimensional (3D) anatomical image data. An example method includes analyzing a three-dimensional (3D) data set of one or more medical images using an artificial intelligence (Al) model to identify two-dimensional (2D) regions of interest in the 3D data set having an increased probability of a diagnostic state, and providing the regions of interest to another process for additional analysis. In some embodiments, said analyzing the 3D data set includes generating a 3D heat map, a 3D segmentation, or both, of the 3D data set using the Al model, and identifying the regions of interest using another Al model, wherein each region of the regions of interest includes a respective 2D data set.

### CROSS-REFERENCE TO RELATED APPLICATIONS

This application claims priority to U.S. Provisional Application No. 63/481,761, filed Jan. 26, 2023, which application is hereby incorporated by reference, in its entirety, for any purpose.

### STATEMENT REGARDING RESEARCH & DEVELOPMENT

This invention was made with government support under Grant No. R01 EB031002, awarded by the National Institutes of Health, and Grant No. DGE-1762114, awarded by the National Science Foundation. The government has certain rights in the invention.

### TECHNICAL FIELD

This application relates to the analysis of medical images. Examples of identifying regions of interest in three-dimensional medical image data are described, including the use of artificial intelligence models.

### BACKGROUND

Medical image analysis can be complex. Modern medical imaging techniques may generate large datasets representing 3D images of patient anatomy. It is infeasible for clinical practitioners to review the entirety of these datasets in many instances. For example, medical practitioners may review only a limited set of images generated from a full three-dimensional tissue biopsy. This kind of limited review is detrimental for accurately diagnosing disease or another anomalies present in medical images.

As an example, a process of review of esophageal samples for neoplasia is described. The incidence of esophageal adenocarcinoma (EAC) has been rising dramatically in Western populations over the last several decades. Most EAC patients are diagnosed with late-stage or distant disease for which the five-year survival rate is less than 20%. EAC is believed to arise from Barrett's esophagus (BE), a complication of gastrointestinal reflux disease that results in esophageal intestinal metaplasia. BE typically progresses to low-grade dysplasia (LGD) and then high-grade dysplasia (HGD) before finally advancing to EAC. While the risk of progression to EAC is low for patients with BE (0.3% per year), the risks of progression to EAC increase for patients who have developed LGD and HGD to 2% and over 6% per year, respectively. Therefore, patients with BE are recommended to receive periodic endoscopic screening for neoplasia, such as dysplasia or EAC, for early detection and early intervention that is key to maximizing patient outcomes. During these screening procedures, 4-quadrant random biopsies are obtained via endoscopic forceps at 1-2 cm increments along the segments of the esophagus containing BE, as well as of suspicious areas seen with endoscopy. In addition to the sampling limitations of random biopsies, the gold-standard method for evaluating these biopsies, slide-based histology, also severely undersamples the biopsied tissues. In conventional histology, biopsy specimens are thinly sectioned, mounted onto glass slides, and stained with hematoxylin and eosin (H&E) to enable microscopic evaluation by pathologists. Since this process is destructive to the tissue, only a few tissue sections, typically 4-16 sections, are processed as H&E slides. The limited amount, such as less than 1%, of each biopsy that pathologists view as two-dimensional (2D) sections may negatively impact the sensitivity for detecting neoplasia.

Early detection of esophageal neoplasia in patients with BE is key to increasing a chance of patient survival. However, histologic evaluation of biopsies obtained during regular surveillance endoscopy of BE patients is not ideal for detecting neoplasia since only a limited number of thin tissue sections from a relatively small number of biopsies is examined. The comprehensive evaluation of whole biopsies with three-dimensional (3D) pathology may improve the detection of neoplasia in patients with BE, but large 3D pathology datasets can be tedious for pathologists to analyze.

While the example of neoplasia detection in BE patients has been described, analogous challenges are present in the review and diagnosis of other diseases based on medical images and/or tissue biopsies.

### SUMMARY

Examples of methods are described herein. An example method includes analyzing a 3D data set of one or more medical images using an artificial intelligence (AI) model to identify 2D regions of interest in the 3D data set having an increased probability of a diagnostic state, and providing the regions of interest to another process for additional analysis.

In some example methods, said analyzing the 3D data set include generating a 3D heat map, a 3D segmentation, or both, of the 3D data set using the AI model, and identifying the regions of interest using another AI model. In some examples each region of the regions of interest include a respective 2D data set. In some examples, the AI model includes either a deep learning model or a patch-based neural network and the another AI model includes a machine learning classifier. In some examples, the AI model includes at least one of convolutional neural networks (CNNs) or vision transformers (ViTs). In some examples, the another AI model includes at least one of support vector machines (SVM), RFCs, or k-means classifiers.

In some example methods, the machine learning classifier is trained using features that account for a difference between smaller focal areas and larger or diffuse areas. In some examples, the features include a ranking of predicted probability of a patch within an image, a number of patches having probability over a threshold, and a standard deviation of probabilities of patches in the image.

In some example methods, the diagnostic state is neoplasia. In some examples, the neoplasia is esophageal neoplasia. In some examples, the neoplasia is prostate cancer.

In some example methods, the one or more medical images include an image of an esophageal specimen. In some examples, the one or more medical images include an image of a prostate specimen.

Examples of systems are described herein. An example system that receives a 3D data set of one or more medical images includes one or more processors. The one or more processors analyze the 3D data set of one or more medical images using an AI model to identify 2D regions of interest in the 3D data set having an increased probability of a diagnostic state, and further provide the 2D regions of interest to another process for additional analysis.

In some example systems, the one or more processors generate a 3D heat map, a 3D segmentation, or both, of the 3D data set using the AI model, and further identify the regions of interest using another AI model. In some examples, each region of the regions of interest includes a respective 2D data set. In some examples, the AI model includes a patch-based neural network and the another AI model includes a machine learning classifier. In some examples, the AI model includes at least one of CNNs or ViTs. In some examples, the another AI model includes at least one of SVMs, RFCs, or k-means classifiers. In some examples, the machine learning classifier is trained using features that account for a difference between smaller focal areas and larger or diffuse areas. In some examples, the features include a ranking of predicted probability of a patch within an image, a number of patches having probability over a threshold, and a standard deviation of probabilities of patches in the image.

Examples of non-transitory computer readable media encoded with instructions are described herein. An example of non-transitory computer readable medium encoded with instructions which, when executed, cause a system to perform operations including analyzing a 3D data set of one or more medical images using an AI model to identify 2D regions of interest in the 3D data set having an increased probability of a diagnostic state, and providing the 2D regions of interest to another process for additional analysis.

In some example non-transitory computer readable media, said analyzing the 3D data set includes generating a 3D heat map, a 3D segmentation, or both, of the 3D data set using the AI model, and identifying the regions of interest using another AI model. In some examples, each region of the regions of interest includes a respective 2D data set. In some examples, the AI model includes at least one of CNNs or ViTs. In some examples, the another AI model includes at least one of SVMs, RFCs, or k-means classifiers. In some examples, the AI model includes either a deep learning model or a patch-based neural network and the another AI model includes a machine learning classifier. In some examples, the machine learning classifier is trained using features that account for a difference between smaller focal areas and larger or diffuse areas.

### BRIEF DESCRIPTION OF THE DRAWINGS

FIG. 1 is a schematic illustration of a computer system that may be used to implement systems and methods in accordance with examples described herein.

FIG. 2 illustrates an example flowchart for a method for identifying 2D regions from 3D anatomical image data in accordance with examples described herein.

FIGS. 3A and 3B illustrate example motivation for and advantages of AI-triaged 3D pathology in accordance with examples described herein.

FIG. 4 illustrates an example flowchart of a method for analyzing 3D data set in accordance with examples described herein.

FIG. 5 is a schematic illustration of endoscopic screening with biopsies evaluated by conventional histology and non-destructive 3D pathology in accordance with examples described herein.

FIGS. 6A-6F illustrate an example prediction pipeline for AI-triaged 3D pathology in accordance with examples described herein.

FIGS. 7A-7E illustrate performance of patch-based and image-based classification in accordance with examples described herein.

FIG. 8A an 8B illustrate a preliminary clinical validation study in accordance with examples described herein.

FIG. 9 shows a comparison table listing an independent validation cohort of the twenty endoscopic biopsies evaluated with conventional 2D histology and AI-triaged 3D pathology in accordance with examples described herein.

### DETAILED DESCRIPTION

Certain details are set forth herein to provide an understanding of described embodiments of technology. However, other examples may be practiced without various of these particular details. In some instances, well-known computer system components, artificial intelligence (AI) techniques, imaging particulars, sample acquisition techniques, circuits, control signals, timing protocols, and/or software operations have not been shown in detail in order to avoid unnecessarily obscuring the described embodiments. Other embodiments may be utilized, and other changes may be made, without departing from the spirit or scope of the claims.

While examples are described herein in the context of esophageal specimens, esophageal biopsies and/or esophageal neoplasia, it is to be understood that other tissue or medical images may be analyzed using techniques described herein, and that systems and techniques described herein may be used to identify other diagnostic states instead of or in addition to neoplasia and/or esophageal neoplasia.

Examples of a deep learning-based method to automatically identify image sections of interest within 3D pathology datasets for pathologist review are described herein. For example, 2D image sections may be identified that have an increased and/or highest risk of disease or other anomaly. An AI model may be used to identify the image sections from within the 3D dataset for further analysis. An example method may generate a 3D heat map indicative of risk within a medical image. The 3D heat map may be generated using an AI model. The example method may identify image sections of interest from the 3D heat map. The image sections of interest may be identified in some examples using another AI model. In some examples, the image sections of interest are 2D sections. In some examples, the image sections of interest are 3D sections, which may be thinner sections than the full 3D image. The thin 3D sections of interest may, for example, be thin enough for a medical practitioner to visually review. The identified sections may be provided to another process for additional analysis. For example, the identified sections may be provided to a computer system for display and review by a pathologist or other clinician. In this manner, a workload presented to the next process (e.g., the pathologist review) may be reduced. The process may be utilized only to analyze suggested sections of the 3D dataset, and those sections may be those which are indicative of an increased and/or highest risk of disease or other anomaly.

In a particular example of identifying neoplasia, open-top light-sheet (OTLS) microscopy was used to obtain 3D pathology datasets of archived (e.g., formalin-fixed paraffin-embedded) esophageal biopsies. An example method first generates a 3D heat map indicating neoplastic risk within each biopsy with a patch-based sensitivity greater than a threshold sensitivity (e.g., 90%) with an area under the receiver operating characteristic (ROC) curve (AUC) that is about a predetermined value (e.g., 0.89). The example method identifies 2D image sections from the 3D heat map that are most likely to contain neoplasia with an AUC (for example, approximately 0.92). A preliminary clinical validation study was performed with twenty biopsies to compare AI-triaged 3D pathology with the conventional slide-based histopathology. In the AI-triaged 3D pathology, three image sections were selected per biopsy for twenty biopsies with the highest risk for neoplasia for pathologist review. In the slide-based histopathology, up to sixteen tissue sections per biopsy were selected for the same twenty biopsies. Among the twenty biopsies evaluated, three biopsies diagnosed as benign by the conventional histology were found to contain neoplasia based on AI-triaged 3D pathology. These results demonstrate that AI-triaged 3D pathology has the potential to improve disease detection while reducing pathologist workloads for neoplasia risk assessment by reviewing smaller samples from biopsies.

In other examples, examples of methods described herein to identify the image sections of interest within large 3D pathology datasets for pathologist review may be used for prognostic Gleason grading of prostate biopsies. For example, patients with low-grade prostate cancer, such as Gleason grade group (GG) 1 may be candidates for active surveillance, while patients with GG 2 and above typically receive curative therapy (radiation and/or surgery). The accurate grading of prostate cancer by histologic evaluation of biopsies may be hindered by a limited number of thin tissue sections (equivalent to ~1% of the biopsy) that are visualized. Comprehensive 3D pathology may improve detection of relatively aggressive prostate cancer (GG 2 and above); however the large datasets are tedious to manually review and analyze. Classification of image sections within 3D prostate datasets as containing GG 1 vs. GG 2 prostate cancer may be performed by methods described herein, and identified image sections (e.g., highest-risk image sections) may be selected for pathologist review. Thus, accurate and time-efficient grading of prostate cancer may be achieved.

Accordingly, examples described herein include methods of using AI models to identify 3D or 2D regions of interest within 3D pathology datasets. The identified regions of interest may be presented to another process for further analysis, which may reduce the workload for that additional reviewing process. For example, the identified regions of interest may be provided to a computer system for display. A pathologist may review the identified and displayed regions manually, with the intention of improving diagnostic determinations for patients and/or to reduce pathologist workloads. The 3D pathology dataset may comprehensively sample the tissue. The 3D pathology data set may be acquired by any of a variety of microscopy technologies (e.g., confocal, multiphoton, light-sheet, or micro CT). The tissues being examined may be surgical specimens or biopsies from generally any tissue, such as any organ. The samples may be analyzed for generally any disease or anomaly, such as, but not limited to, esophageal neoplasia or prostate cancer.

Accordingly, examples described herein include computational methods of identifying image sections of interest (which may be referred to as “levels”) within a 3D pathology dataset. The identified sections may be provided for pathologists to manually examine, with the intention of improving diagnostic determinations for patients and/or to reduce pathologist workloads.

In some examples, an AI model is provided for triage of 3D pathology datasets. The model may be trained with detailed annotations provided by a pathologist, whether at the pixel-level, image-level, biopsy/specimen-level, or with patient-level labels.

In some examples, an AI model may be trained in a fully supervised manner to predict which image sections are selected.

In some examples, a two-step process may be used in which one AI model is used to generate a 3D heat map or 3D segmentation of a dataset. The heat map may generally reflect a probability of disease or anomaly at each location of the heat map. A second AI model may be trained to identify the regions of interest that are selected for pathologists to view. The regions of interest may be 2D regions or thin 3D regions in some examples. The regions of interest may be the regions of increased and/or highest risk of disease or another anomaly in some examples.

In some examples of such a two-step process, in the first step, a deep-learning (DL) patch-based classifier (e.g., Resnet) may be used. The DL model may be trained with pixel-level annotations on a training data set in some examples. In the second step, a random forest classifier (RFC) may be used. The RFC may be trained with image-level annotations on the training data set.

In some examples, a set of specific features may be used to train an RFC for image-level diagnosis (the second step), in which the RFC learns to account for smaller focal areas of high risk as well as larger or diffuse areas of low-to-moderate risk in datasets.

In some examples, with 2D image-level annotations, AI models may be trained in a weakly supervised manner to predict which 2D image levels (or 2D regions of interest) are selected for pathologists to view.

In some examples, with biopsy/specimen-level annotations, AI models may be trained in a weakly supervised manner to predict which 2D image levels, 2D regions of interest, or 3D regions of interest are selected for pathologists to view.

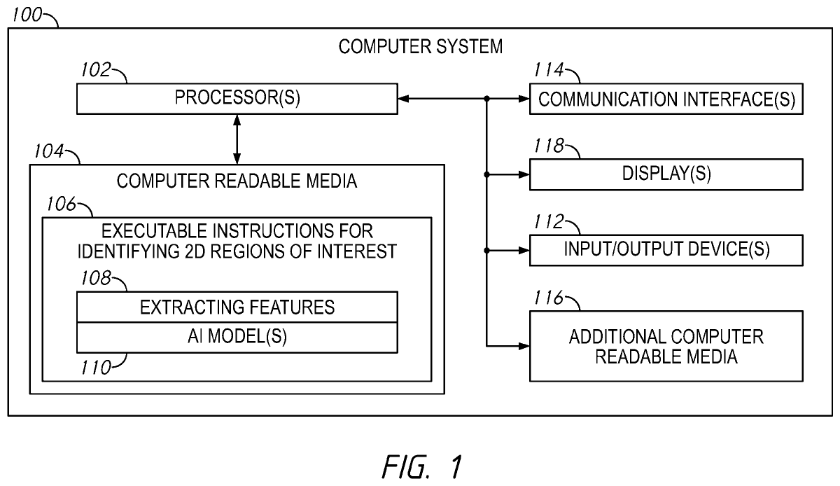

FIG. 1 is a schematic illustration of a computer system 100 that may be used to implement systems and methods in accordance with examples described herein. The computer system 100 includes one or more processor(s) 102, one or more computer readable media 104, executable instructions for identifying 2D regions of interest 106 including executable instructions for extracting features 108 and executable instructions for executing and/or training one or more AI model(s) 110. The computer system 100 may further include an input/output device(s) 112, communication interface(s) 114, one or more additional computer readable media 116, and one or more display(s) 118.

The components shown in FIG. 1 are exemplary. Additional, fewer, and/or different components may be included in other examples.

The computer system 100 may be implemented, for example, using one or more computers, servers, medical devices, smart phones, smart devices, tablets, and/or appliances. The computer system 100 may be coupled to and/or in communication with a source or storage of image data, such as one or more 3D data sets described herein. The computer system 100 may identify regions of interest in the 3D data sets, which may be further analy zed by another process (e.g., another computer system and/or a human reviewer). The computer system 100 of FIG. 1 includes one or more processor(s) 102 and one or more computer readable media 104. The computer readable media 104 may include executable instructions for identifying 2D regions of interest 106. In some embodiments, the computer system 100 may be physically coupled to a source of the 3D image data, such as a microscope or other imaging system. In some examples, the computer system 100 may include input devices and/or output devices 112 that may generate and/or provide the 3D image data, such as a microscope or other imaging system. In other embodiments, the computer system may not be physically coupled to a source of the 3D image data but may be in communication with a source of the 3D image data. In some examples, the computer system 100 may include communication interface(s) 114 that may be in communication with a source of the 3D image data, such as a microscope or other imaging system, or with storage containing one or more 3D image data sets.

Computer systems, such as computer system 100 of FIG. 1, may include one or more processor(s) 102. Any kind and/or number of processors may be present, including one or more central processing unit(s) (CPUs), graphics processing units (GPUs), other computer processors, mobile processors, digital signal processors (DSPs), field programmable gate arrays (FPGAs), application specific integrated circuits (ASICs), microprocessors, computer chips, and/or processing units configured to execute machine-language instructions and process data, such as executable instructions for identification of 2D regions of interest.

Computer systems, such as the computer system 100 of FIG. 1, may further include computer readable media 104. Any type or kind of media may be present, including memory and/or storage. Examples include read only memory (ROM), random access memory (RAM), solid state drive (SSD), secure digital card (SD card), hard drive, network-attached storage, etc. Computer systems, such as the computer system 100 of FIG. 1, may further include additional computer readable media 116. While each single box is depicted as computer readable media in FIG. 1, any number of memory and/or storage devices may be present. The computer readable media, such as the computer readable media 104 and/or the additional computer readable media 116 may be in communication with (e.g., electrically connected to) the processor(s) 102.

The computer readable media 104 may store executable instructions for execution by the processor(s), such as executable instructions for identifying 2D regions of interest 106. The executable instructions for identifying 2D regions of interest 106 may include executable instructions for extracting features 108 from one or more 3D data sets, including features described herein. While the figure refers to identifying 2D regions of interest, it is to be understood that in some examples the regions of interest may have three-dimensions, such as a thin 3D region. Generally, the thin 3D region may be smaller in one dimension than the full 3D dataset, typically significantly smaller.

The executable instructions for identifying 2D regions of interest 106 may include instructions for executing and/or training one or more AI model(s) 110. In some examples, training of the AI model(s) and use of the AI models to identify regions of interest may be performed using a same computer system, such as the computer system 100 of FIG. 1. In other example, one or more of the AI model(s) may be trained using a different computer system, and data encoding the trained AI model(s) 110 may be stored in the computer readable media 104 of the computer system 100 of FIG. 1 and may be used to identify 2D regions of interest.

Training of the AI model(s) may be performed using any of a variety of techniques including, but not limited to, supervised learning, unsupervised learning, clustering, and/or reinforcement learning.

In some examples, the AI model(s) may be implemented using one or more machine classifiers, such as one or more DL models, neural networks (e.g., patch-based neural networks), and/or machine learning models, including but not limited to one or more decisions trees. Examples of machine learning models may include convolutional neural networks (CNNs), vision transformers (ViTs), support vector machines (SVM) (e.g., radial basis function (RBF) kernel SVM), RFCs, and/or a k-means classifier.

In some examples, multiple AI models may be used to identify 2D regions of interest. For example, a first AI model may generate a 3D heat map and/or 3D segmentation based on an input 3D data set. This first AI model may, for example, be implemented using a DL model and/or a patch-based neural network. In some examples, the first AI model may, for example, uses multiple classifiers. Such classifiers may include, but are not limited to, CNNs, and recently developed ViTs. A second AI model may identify 2D regions of interest based on the 3D heat map and/or 3D segmentation. The second AI model may be implemented, for example, using a machine learning model such as an SVM, RFC, and/or a k-means classifier.

Note that, in some examples, the first AI model may be trained using pixel-level annotations of 3D training data sets identifying regions of interest. In some examples, the pixel-level annotations of 3D training data sets may be provided by a medical expert (e.g., a pathologist). In some examples, the second AI model may be trained using yes or no annotations indicating whether there is disease in each image of a training set of 2D images. In some examples, such annotations may be labeled by a medical expert (e.g., a pathologist).

In this manner, to identify 2D regions of interest, one or more 3D data sets of image data may be received by the computer system. The image data may generally be of any medical system, including generally of any tissue. In some examples, such tissue may be esophageal tissue as described in examples herein. A first AI model may be used to generate a heat map and/or segmentation of the 3D data sets relating to regions of interest for a particular diagnostic state (e.g., neoplasia as described in examples herein). In some examples, the heat map may be a risk map in which each pixel, each segment, each block, and/or each region in the risk map may be weighted with probability of being disease or other anomaly. A second AI model may be used to identify 2D regions of interest based on the heat map and/or segmentation.

The computer system 100 of FIG. 1 may include additional components, not all of which are necessarily depicted in FIG. 1. Examples of additional components may include one or more communication interface(s) 114. In some examples, the one or more communication interface(s) 114 may include wireless communication interfaces such as a WiFi, Bluetooth, network interface, cellular interface, wired communication interfaces such as serial buses (e.g., universal serial bus) or parallel data interface, and/or other communications interface. The communication interface(s) 114 may be used to receive one or more 3D data sets in some examples. The communication interface(s) 114 may be used to provide (e.g., transmit) an identification of 2D regions of interest and/or data corresponding to the 2D regions of interest to another computer system for review by another process (e.g., by a human reviewer, such as a medical expert). The computer system 100 may include one or more display(s) 118. The display(s) 118 may be used, for example, to display 3D data sets, 2D regions of those datasets (including regions of interest), and/or heat maps or segmentations of 3D data sets. The computer system 100 may include one or more input and/or output devices 112 including, but not limited to, one or more touchscreens, mice, keyboards, cameras, and/or printers. The input devices may be operated by a user of the computer system 100 to provide commands and/or data to the computer system 100. The computer system 100 may include and/or be in communication with additional computer readable media 116 that may provide temporary data to be used during processing, or permanent data to be stored for record or presented to a user.

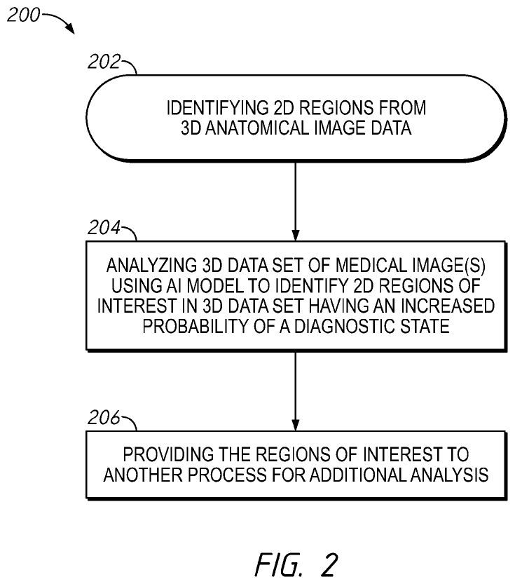

FIG. 2 illustrates an example flowchart of a method 200 for identifying 2D regions from 3D anatomical image data 202 in accordance with examples described herein. In some examples, the method 200 may be performed by the computer system 100 of FIG. 1. Although the example method 200 depicts a particular sequence of operations, the sequence may be altered without departing from the scope of the present disclosure. For example, some of the operations depicted may be performed in parallel or in a different sequence that does not materially affect the function of the method 200. In other examples, different components of an example device or system that implements the method 200 may perform functions at substantially the same time or in a specific sequence. For instance, in some examples, the 2D regions to be identified may be 2D regions of interest that are selected (e.g., most important) for pathologists to view. In other examples, instead of 2D regions, 2D levels or 3D regions of interest that are relatively thin slices with less thickness may be identified using the similar sequence of operations without departing from the scope of the present disclosure. Thus, the flowchart of the method 200 demonstrates how AI models may be used to identify 3D or 2D regions of interest within 3D pathology datasets for pathologists to review manually.

According to some examples, the method 200 includes operation 204 analyzing 3D data set of medical image(s) using AI model to identify 2D regions of interest in 3D data set having an increased probability of a diagnostic state and operation 206 providing the regions of interest to another process for additional analysis.

In some examples, the operation 204 may be performed by the one or more processor(s) 102 of the computer system 100. The one or more processor(s) 102 may analyze the 3D data set of medical images by performing executable instructions for identifying 2D regions of interest 106 stored in the computer readable media 104 of the computer system 100. For example, the one or more processor(s) 102 may receive 3D data sets, such as 3D pathology datasets of archived (e.g., formalin-fixed paraffin-embedded) biopsies. The 3D pathology dataset may comprehensively sample the tissue, unlike slide-based 2D visualization. The 3D pathology data set may be acquired by any of a variety of microscopy technologies (e.g., confocal, multiphoton, light-sheet, or micro CT). The tissues being examined may be surgical specimens or biopsies from any organ or disease, not limited to neoplasia, such as esophageal neoplasia or prostate cancer.

The executable instructions for identifying 2D regions of interest 106 performed in the operation 204 may include executable instructions for extracting features 108 from one or more 3D data sets, including features described herein.

The executable instructions for identifying 2D regions of interest 106 may additionally or instead include instructions for executing and/or training one or more AI model(s) 110. An AI model is provided for triage of 3D pathology datasets. In some examples, training of the AI model(s) and use of the AI models to identify regions of interest may be performed using a same computer system, such as the computer system 100 of FIG. 1. In other examples, the one or more of the AI model(s) 110 may be trained using a different computer system, and data encoding the trained AI model(s) 110 may be stored in the computer readable media 104 of the computer system 100 of FIG. 1 and may be used to identify 2D regions of interest. Training of the AI model(s) may be performed using any of a variety of techniques including, but not limited to, supervised learning, unsupervised learning, clustering, and/or reinforcement learning. In some examples, AI models may be trained in a fully supervised manner to predict which image patches are selected (e.g., most important) for pathologists to view. In some examples, the AI model(s) may be implemented using one or more machine classifiers, such as one or more DL models, neural networks (e.g., patch-based neural networks), and/or machine learning models, including but not limited to one or more decisions trees. Examples of machine learning models may include CNNs, ViTs, SVM (e.g., RBF kernel SVM), RFCs, and/or a k-means classifier.

In block 206, the regions of interest may be provided to another process for additional analysis. This may include, for example, providing the regions of interest to another computer system. The computer system may, for example, display the regions of interest for review by a clinician. In some examples, other processes may be used to provide additional analysis.

Examples described herein include computational methods of identifying particular (e.g., highest and/or increased risk) 2D image sections (which may be referred to as “levels”) within a 3D pathology dataset for pathologists to manually examine. The model may be trained with detailed annotations provided by a pathologist, whether at the pixel-level, image-level, biopsy/specimen-level, or with patient-level labels. In some examples, with 2D image-level annotations, AI models may be trained in a weakly supervised manner to predict which 2D image levels (or 2D regions of interest) are selected (e.g., most important) for pathologists to view. In some examples, with biopsy/specimen-level annotations, AI models may be trained in a weakly supervised manner to predict which 2D image levels, 2D regions of interest, or 3D regions of interest are selected (e.g., most important) for pathologists to view. In some examples, with patient labels (patient outcomes), AI models may be trained in a weakly supervised manner to identify the 2D levels, 2D regions of interest, or 3D regions of interest that are selected (e.g., most important) for pathologists to view. According to some examples, the method 200 includes providing the regions of interest to another process for additional analysis at operation 206. For example, a number of image sections with the highest risk for neoplasia per biopsy used for the 3D heat map or segmentations generation may be as few as three for pathologist review. Thus, the method 200 for identifying 2D regions from 3D anatomical image data 202 may improve diagnostic determinations for patients and/or reduce pathologist workloads.

Following the additional analysis in block 206, a disease or anomaly may be identified based on the additional analysis. Accordingly, analysis of identified regions of interest in a 3D dataset may be used to identify a disease and/or anomaly in the imaged tissue sample and/or anatomy. Based on the identification of disease and/or anomaly, treatment modalities may be determined and/or adjusted.

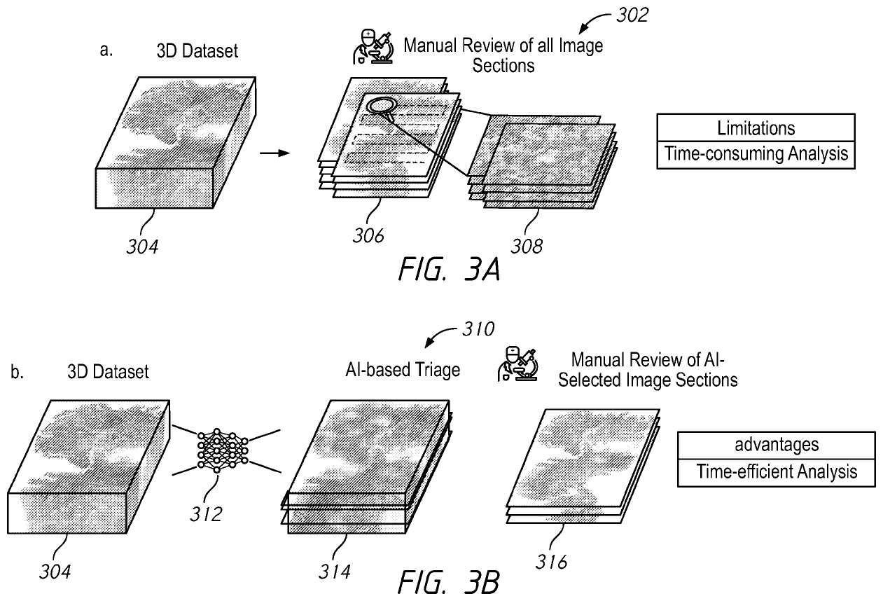

FIGS. 3A and 3B illustrate example motivation for and advantages of AI-triaged 3D pathology in accordance with examples described herein. FIG. 3A illustrates non-destructive 3D pathology 302 which provides comprehensive sampling of biopsies as 3D pathology datasets 304 while preserving the tissue for downstream assays/archives. However, the manual evaluation of all image sections 306 including region(s) of interest 308 potentially including neoplasia in such large 3D pathology datasets 304 of comprehensive sampling of biopsies would be time-consuming.

FIG. 3B illustrates an example AI-based triage method 310 in accordance with examples described herein. In the AI-based triage method 310, automatic identification of regions most likely to contain neoplasia within the 3D pathology datasets 304 may be performed by executing AI model(s) 312, such as the operation 204 by generating 3D heat maps 314. Based on the identified 2D or 3D regions, 2D image sections 316 (e.g., three sections per biopsies) with the highest probability of containing neoplasia may be selected for manual review by a pathologist. Thus, the workload for pathologists may be reduced in comparison to conventional histology as shown in FIG. 3A.

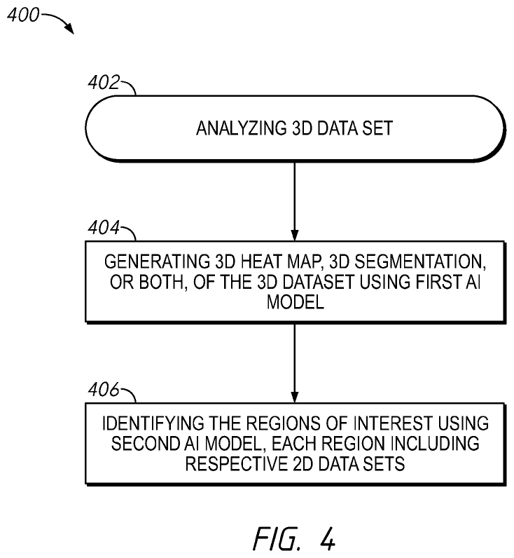

FIG. 4 illustrates an example flowchart of a method 400 for analyzing 3D data set 402 in accordance with examples described herein. In some examples, analyzing 3D data set 402 may be performed as the operation 204 of FIG. 2 by the computer system 100 of FIG. 1. In some examples, multiple AI models may be used to identify 2D regions of interest by analyzing the 3D data set. Although the example flowchart depicts a particular sequence of operations, the sequence may be altered without departing from the scope of the present disclosure. For example, some of the operations depicted may be performed in parallel or in a different sequence that does not materially affect the function of the routine. In other examples, different components of an example device or system that implements the routine may perform functions at substantially the same time or in a specific sequence. To identify 2D regions of interest, one or more 3D data sets of image data may be received by a computer system, such as the computer system 100 of FIG. 1. The image data may generally be of any medical system, including generally of any tissue. In some examples, such tissue may be esophageal tissue or prostate tissue as described in examples herein. In some examples, a two-step process may be used in which a first AI model generates a 3D heat map or 3D segmentation of selected (e.g., most important) regions (e.g., regions at highest risk for malignancy) based on the 3D data sets in operation 206, and then a second AI model trained to identify the 2D levels or 2D regions of interest selects (e.g., most important) for pathologists to view.

According to some examples, the method 400 includes generating a 3D heat map, 3D segmentations, or both, of the 3D dataset using a first Al model at operation 404. The one or more processor(s) 102 may generate either 3D heat map or 3D segmentations of the 3D dataset weighted with probability of being disease. The heat map and/or segmentations of the 3D data set may be related to regions of interest for a particular diagnostic state (e.g., esophageal neoplasia, or prostate cancer as described in examples herein). In some examples, the heat map may be a risk map in which each pixel, each segment, each block, or each region in the risk map may be weighted with probability of being disease. Thus, the 3D heat map or the 3D segmentations may indicate a neoplastic risk within each biopsy based on the extracted features of the 3D pathology datasets. The first AI model may identify a 3D heat map and/or 3D segmentation based on an input 3D data set. In some examples, the first AI model may be included in the one or more AI model(s) 110. The first AI model may, for example, be implemented using a DL model and/or a patch-based neural network. In some examples, the first AI model may, for example, use multiple classes. Such classes may include, but are not limited to, CNNs and recently developed ViTs. In some examples, a DL patch-based classifier (e.g., Resnet) may be used for generating the 3D heat map, 3D segmentation, or both, of the 3D dataset. In some examples, the first AI model is a DL model. In some examples, the first Al model may be trained with pixel-level annotations of 3D training data sets. In some examples, the pixel-level annotations may be provided by a medical expert (e.g., a pathologist). For example, the pixel-based annotations may be provided by pathologists circling regions of interest in an image. Using the first AI model trained as described herein, the heat map and/or segmentation of the 3D data sets may be related to regions of interest for a particular diagnostic state (e.g., esophageal neoplasia, or prostate cancer as described in examples herein).

According to some examples, the method 400 includes identifying the regions of interest using a second AI model at operation 406. In some examples, each region of the regions of interest may include respective 2D data sets. The second AI model may identify 2D regions of interest based on the 3D heat map and/or 3D segmentation. The second AI model may be implemented, for example, using a machine learning model such as an SVM, an RFC, and/or a k-means classifier. In some examples, the second AI model, such as the RFC, may be trained with image-level annotations. For example, the image-level annotations may use yes or no annotations indicating whether there is disease in each image of a training set of 2D images. In some examples, such annotations may be labeled by a medical expert (e.g., a pathologist). For example, the second AI model, such as a machine learning classifier may be trained using weakly-supervised training where the pathologist just labels an image, indicating whether any disease is observed in this image. In some examples, a set of specific features may be used to train an RFC for image-level diagnosis (the second step), in which the RFC learns to account for smaller focal areas of high risk as well as larger or diffuse areas of low-to-moderate risk in OTLS datasets. In some examples, the set of specific features may include a ranking of predicted probability of a patch within an image, a number of patches having probability over a threshold, and a standard deviation of probabilities of patches in the image. Thus, using the second AI model at operation 406, 2D image sections from the 3D heat map that are most likely to contain neoplasia (for example, with an AUC approximately 0.92) may be identified. Based on the 3D heat map or segmentations, the second AI model may provide fewer 2D image sections (e.g., three 2D image sections) for pathologist review.

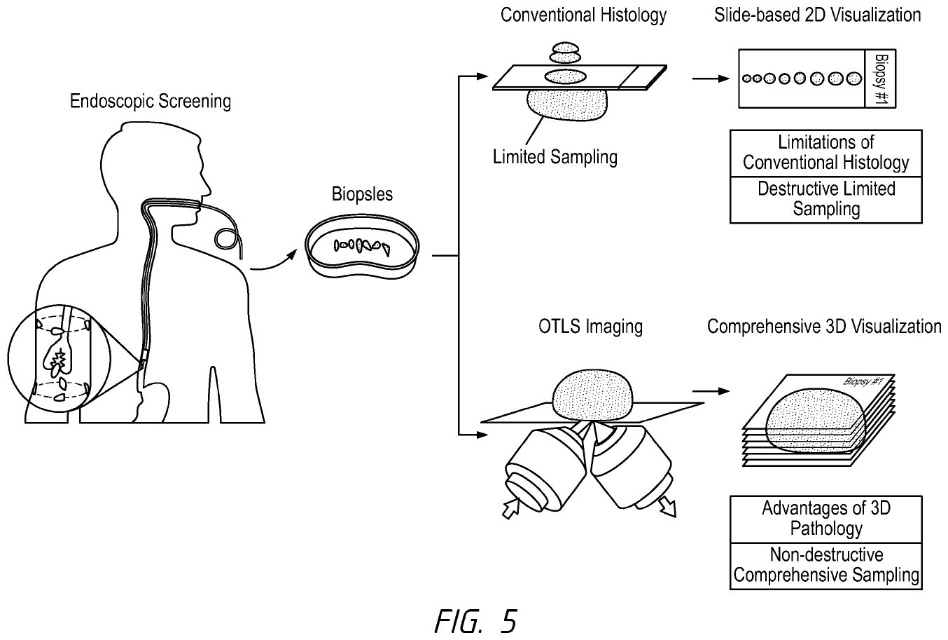

FIG. 5 is a schematic illustration of endoscopic screening with biopsies evaluated by conventional histology and non-destructive 3D pathology in accordance with examples described herein. During periodic endoscopic screening of BE patients, 4-quadrant biopsies are obtained at ~1 cm increments along the length of the BE (Seattle protocol). With conventional histology, these biopsies are thinly sectioned, mounted onto glass slides, and stained with H&E for pathologist review, in which only a small fraction, such as less than 1%, of each biopsy may be examined. In this study, OTLS microscopy is used to comprehensively image the whole biopsy in 3D without requiring destructive tissue sectioning.

Although OTLS microscopy is a non-destructive approach that enables pathologic evaluation of whole biopsies, 3D pathology datasets can be tedious to assess as described herein. Considering that endoscopies alone generate 20 million cases annually in the U.S. for pathologist review, diagnostic workflows that take advantage of 3D information without further increasing pathologist workloads would be desirable. To this end, examples of AI-based triage methods are described herein that may guide and streamline the diagnosis of 3D pathology datasets. Example methods use a DL algorithm to automatically identify neoplastic regions in small 2D image patches, aggregates these patch-based predictions in 3D, and then use an RFC to select certain (e.g., the most important) 2D image sections from the 3D dataset for pathologist review as described herein. The patch-based and image section-based performance of example triage methods are quantified and a preliminary clinical validation study reported herein showing that AI-triaged 3D pathology can potentially improve the sensitivity of diagnosing neoplasia in endoscopic biopsies while reducing the workload for pathologists in comparison to standard-of-care histology.

### EXAMPLES

### General Framework for Computational Triage Method

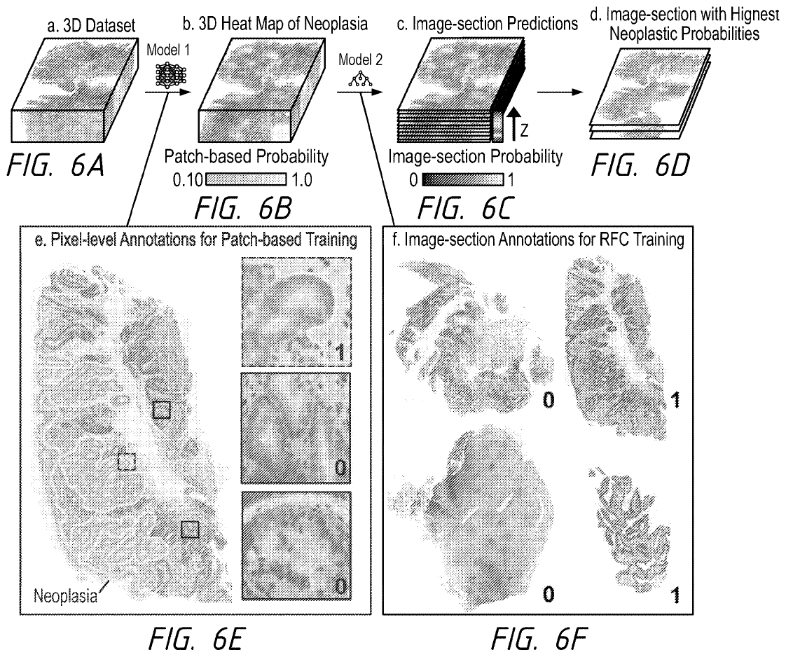

FIGS. 6A-6F illustrate an example prediction pipeline for AI-triaged 3D pathology in accordance with examples described herein.

### Biopsy Specimen Preparation

Each sample to be used for training and performing a method of identifying 2D regions from 3D anatomical image data may be prepared and OTLS imaging may be performed. In some examples, esophageal biopsy and endoscopic mucosal resection (EMR) specimens were obtained as formalin-fixed paraffin-embedded (FFPE) blocks from the Gastrointestinal Center for Analytic Research and Exploratory Science (GiCaRes) at the University of Washington Medical Center (UWMC). In some examples, archived esophageal specimens (a number of samples n=30) from BE patients were used for the development of a computational triage model. For initial model development, thirty specimens from eleven patients (twenty-two biopsies and eight EMR specimens) were used for algorithm training. Another twenty biopsy specimens from ten other patients were set aside as an independent validation cohort. Specimens were deparaffinized by incubating them at 70° C. for one hour and then immersing them in Xylene at 65° C. for forty-eight hours. Specimens were washed twice in 100% ethanol (EtOH) for one hour each, incubated in 70% EtOH (30% deionized water) for one hour, then stained with a fluorescent analog of H&E by incubating them in 70% EtOH at pH=4 (titrated with HCl) with a 1:1000 dilution of TO-PRO-3 and a 1:2000 dilution of Eosin-Y. Finally, the specimens were optically cleared in ethyl cinnamate (n=1.56) for four hours.

The esophageal specimens were then placed on a flat index-matched (n=1.56) sample plate (Hivex, 200-μm thick) and imaged with a previously reported OTLS microscope using a 20× objective (NA=0.40). A digitally scanned light sheet (NA ~0.09) was used to illuminate TO-PRO-3 and Eosin-Y at wavelengths of 660 nm and 488 nm, respectively. Fluorescence was collected with a band-pass filter (Semrock FF01-496/LP-25 and Semrock LP02-664RU-25) and 20× objective (Nikon CFI60 TU Plan ELWD), then relayed to an sCMOS camera as the specimen was stage-scanned in XYZ. Images were collected with an isotropic sampling pitch of 0.21 μm/px. Data was compressed during imaging by ~10× with B3D lossless compression.

In FIG. 6A, for each 3D pathology dataset, a DL model was used to identify neoplastic image patches. In this example, ResNet18 was used as the DL network. Patch-based predictions were generated and aggregated over all 2D image sections in each 3D specimen, resulting in a 3D heat map that predicts the presence of neoplasia within the whole specimen (see FIG. 6B). In some examples, a DL network trained to assign patch-based probability for each patch extracted from the 2D image sections may be used. These predictions are volumetrically aggregated, resulting in a 3D heat map of the average predicted probability of each patch containing neoplasia, an example of which is shown in FIG. 6B.

Once a 3D probability heat map was generated, an RFC was used to identify the 2D image sections with the highest probability of containing neoplasia. In FIG. 6C, an RFC predicted the probability that each image section within the heat map contains neoplasia.

In FIG. 6D, the top-ranked image sections with the highest neoplastic probabilities were false-colored to create an H&E-like appearance. These image sections were provided to pathologists for their review and diagnosis.

### Training of Patch-Based Algorithm

### Image Pre-Processing Including Annotations

Using OTLS microscopy, 3D pathology datasets were obtained. The 3D pathology datasets of the thirty esophageal specimens from eleven patients were processed to facilitate patch-based training of a DL network. One to two cross-sectional images (2D) were selected from the 3D pathology datasets of each specimen (a total of forty-three image sections) for pathologist annotation and algorithm training. Prior to pathologist review and annotation, these images were false-colored to mimic an H&E-like appearance and saved in a pyramidal TIFF format. FIG. 6E illustrates example pixel-level annotations for training the first AI model in FIG. 4, such as a patch-based algorithm. These images are split into 100 μm×100 μm patches (insets). In FIG. 6E, the training data used for the ResNet model in this example include the forty-three image sections with pixel-level annotations for neoplasia (left). Pixel-level annotations were provided by a board-certified pathologist (DMR) to indicate regions of neoplasia (dysplasia or cancer) using the Automated Slide Analysis Platform (ASAP) and recorded in an XML file (left).

After annotations were prepared, the images were pre-processed for training the DL algorithm (ResNet18) which was pre-trained on ImageNet. The raw data for both fluorescence channels (TO-PRO-3 and Eosin-Y) were normalized and saved as two channels within an RGB image (as per the conventional image format used for patch-based DL algorithms). The Otsu method was used to threshold and segment the tissue boundaries from the background of the image, and overlapping patches (512×512 px or ~100×100 μm, with 50% overlap between adjacent patches) were extracted from the tissue-containing regions in each 2D image. Finally, each patch is assigned a ground truth label of zero if they are entirely benign, or one if they contain any amount of neoplasia based on the pathologist's pixel-level annotations of the images. This procedure generated approximately 393,000 patches (355,800 benign and 37,300 neoplastic) for training.

### Training

During training of the patch-based algorithm, image transformations were applied to patches at random to improve model performance and reduce overfitting: image rotations, vertical and horizontal flips, brightness and contrast adjustments, and saturation adjustments. A K-fold cross-validation technique was used to train the model and estimate its overall performance, where the annotated image data was split into K=15 folds. For each fold, all image patches from twenty-eight specimens were used for training, and patches from the remaining two specimens were held out for testing. The model was trained over twenty epochs with a batch size of 64 for each of the 15 folds. Training took approximately nine hours for each fold on a workstation equipped with a Nvidia Titan Xp graphics card, 128 GB of RAM, and an Intel Xeon processor (E5-1620 v4 3.5 GHz 4 core).

For each 2D image section, overlapping patch-based predictions were aggregated (overlapping patch regions were averaged) to create a probability heat map, for which the intensity value of any given patch (maximum value of 1.0) represents the predicted probability of that image patch containing neoplasia. With 50% overlap between all adjacent patches, the intensity value of each patch in the heat map was therefore the average of 4 overlapping patch predictions (except for patches at the boundary of the tissue).

### Training of Image-Based RFC

FIG. 6F illustrates example image-section annotations for training the second AI model shown in FIG. 4, such as a machine learning model such as an SVM, an RFC, and/or a k-means classifier, such as an RFC algorithm. In some examples, the second AI model, such as a machine learning classifier may be trained using weakly-supervised training where a pathologist labels each image, indicating whether any disease is observed in each image. In FIG. 6F, to train the RFC, 2D image sections are assigned a ground truth label of zero if they are entirely benign, or one if they contain any amount of neoplasia. The RFC was trained to discriminate between benign and neoplastic 2D image sections based on their corresponding probability heat maps generated by the patch-based classifier. A set of 3 “hand-crafted” features extracted from the heat map served as inputs to the RFC: the maximum predicted probability of neoplasia, the number of patches for which P>0.10, and the image noise (standard deviation of the patch values). To train the classifier for this task, the heat maps corresponding to the forty-three annotated images generated during cross-validation testing of the patch-based algorithm were used. The ground-truth label for each image was zero or one according to DMR's annotations, where images that contained any amount of neoplasia were assigned a label of one and entirely benign images were assigned a label of zero. The 15-fold cross-validation was used to train and evaluate the RFC's performance, with the same train-test splits described for the patch-based classifier.

After training the RFC, the classifier was used to generate predictions on all 2D image sections within each 3D pathology dataset. The output for each image section was a single value (maximum of 1.0) corresponding to the probability that the image section contained neoplasia as shown in FIG. 6C. The sections were sorted based on the probability of containing neoplasia, and then the top image sections were identified for manual review by a pathologist as shown in FIG. 6D.

### Patch-Based and Image-Based Classification Results

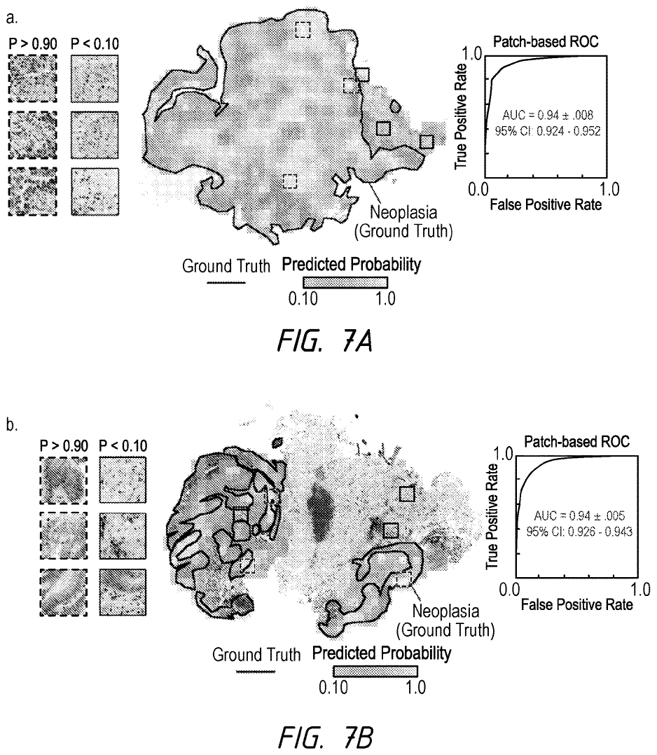

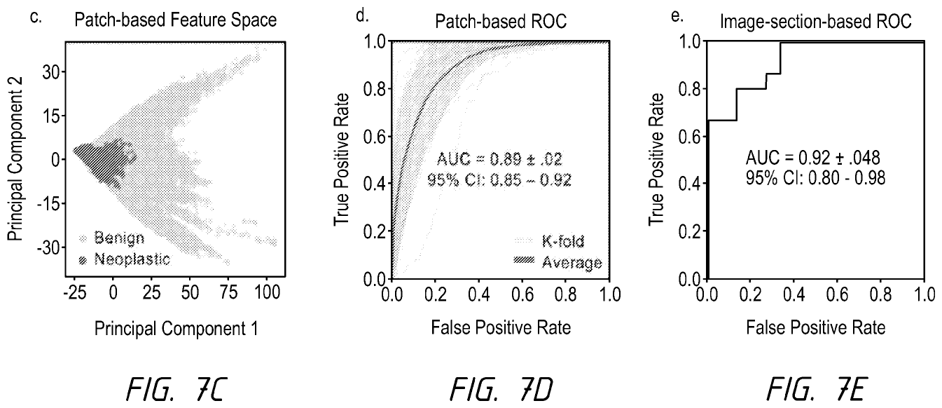

FIGS. 7A-7E illustrate performance of patch-based and image-based classification in accordance with examples described herein.

The performance of an example patch-based DL classifier is demonstrated and described herein. Several examples of 2D heat maps generated by the patch-based classifier are shown along with corresponding ground truth annotations (regions encircled by black lines) in FIGS. 7A and 7B. FIGS. 7A and 7B illustrate probability heat maps generated by an example patch-based DL algorithm during cross-validation testing overlaid onto 2D image sections from 3D pathology datasets of biopsy specimens from the annotated training set. Each heat map value indicates probability of each patch containing neoplasia. The heat maps are overlaid onto their respective H&E false-colored 2D images, and the ground-truth annotations of the neoplastic regions are also shown (regions encircled by black solid lines).

Examples of true-positive and true-negative patch-based predictions from these examples are shown (insets on the left), as well as the average patch-based ROC curve for each of the 2D cross-sectional images. On the left, a few examples of true positive (far left column) and true negative (right column) patch-based predictions are shown. On the right, average patch-based ROC curves are also shown for each image along with AUC values (±94 ). 95% confidence intervals were determined by bootstrapping patch-based predictions (B=10,000). The model's performance was estimated with K-fold cross-validation where K=15.

FIG. 7C shows principal component analysis (PCA) performed on all patch-based predictions for each cross-validation fold to visualize the model's predictions in a feature space. Two principal components were fitted to the 512-dimensional vectors generated by the model for each patch before the final, fully connected layer. Visualization of all predictions in this feature space suggests that the neoplastic patches (solid line) are well-distinguished by the algorithm from benign patches (gray).

In FIG. 7D, ROC curves are plotted for each cross-validation fold for the patch-based DL predictions, as well as for the average of all 15 folds (dark line). The standard deviation is shaded in gray. As shown in FIG. 7D, the overall performance was benchmarked by computing ROC curves for all fifteen cross-validation folds applied to a total of forty-three 2D image sections from thirty specimens. The average ROC curve is also shown (AUC=0.89, σ=0.02, 95% CI: 0.85-0.92) where the 95% confidence interval (CI) is estimated by bootstrapping performance across all cross-validation folds where B=10,000. On average, the algorithm identified neoplastic regions with 90% patch-based sensitivity and 71% patch-based specificity, (e.g., based on the training shown in FIG. 6E), which is deemed adequate for a triage algorithm to screen for the presence of neoplasia in thousands of image patches from hundreds of image sections per 3D pathology dataset.

To demonstrate the performance of the image-based classifier, the RFC's performance at discriminating between image sections that are entirely benign vs. containing any amount of neoplasia was benchmarked. FIG. 7E shows an ROC curve for 2D image section-based predictions (RFC) averaged across all 15 cross-validation folds. The average ROC curve for the performance of this image-based classifier is shown in FIG. 7E, with an AUC=0.92 (σ=0.048, 95% CI: 0.80-0.98). Selection of the most-optimal probability threshold yields an overall 2D image-based sensitivity of 87% and specificity of 73%.

### Preliminary Clinical Validation Study

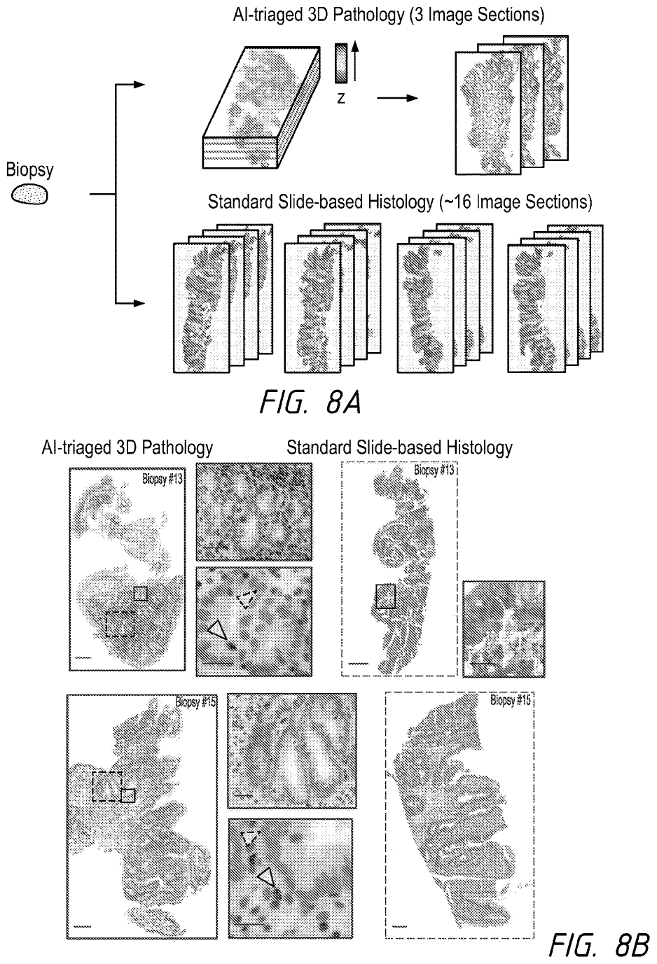

FIG. 8A an 8B illustrate a preliminary clinical validation study in accordance with examples described herein. To explore the clinical value of this triage method, an example AI-triaged 3D pathology method was compared with well-established 2D histology using an independent validation cohort of twenty endoscopic biopsies from ten patients.

FIG. 8A illustrates non-destructive 3D pathology datasets obtained from twenty intact biopsies. The model described herein with an example AI algorithm identified top three image sections with the highest probability of containing neoplasia for pathologist review. The same biopsies were submitted for standard H&E histology, where sixteen or fewer physical tissue sections per biopsy were reviewed. As shown in FIG. 8A, a board-certified gastrointestinal pathologist first diagnosed each biopsy based on the three image sections identified with an example computational triage method described herein. After AI-triaged 3D pathology was performed, conventional histology sections were obtained from the same biopsies. Here, two slides per biopsy of sixteen or less physical tissue sections were viewed by the pathologists. A washout period of at least two weeks was implemented between the review of the AI-triaged three image sections and the review of the standard sixteen histology sections.

FIG. 8B illustrates examples for which AI-triaged 3D pathology upgraded the diagnosis compared with conventional 2D histology. Regions of HGD can be identified in the AI-triaged image sections in biopsy #13 and biopsy #15, as characterized by fused and crowded glands (shown in boxes), large nuclei (also shown in boxes), prominent nucleoli (upper arrowhead), and mitoses (lower arrowhead). However, in the conventional histology sections, architectural features showing basally oriented epithelial cells with mucin caps (shown in box) are consistent with a diagnosis of benign BE. Scale bars are 100 μm for black boxes and 30 μm for all other boxes.

Two examples are shown in FIG. 8B. In the first example (biopsy 13), image sections identified by AI-triaged 3D pathology show hallmarks of HGD such as fused and crowded glands (larger box), large nuclei (smaller box), prominent nucleoli (upper arrowhead), and mitoses (lower arrowhead). However, normal architectural features of basally oriented epithelial cells with mucin caps (box depicted on “standard slide-based histology”) are observed with gold-standard histopathology. In another example (biopsy 15), regions of HGD characterized by focal areas of fused glands (leftmost box), nucleoli (upper arrowhead), and mitosis (lower arrowhead) are seen in the AI-triaged image sections, but are not seen in any of the conventional histology sections.

In addition to the three biopsies that were diagnostically upgraded from benign BE to neoplastic (biopsies 11, 13, and 15), a fourth biopsy diagnosed as LGD by conventional histology (biopsy 2) was upgraded to HGD with AI-triaged 3D pathology. There were no examples of diagnostic downgrading based on AI-triaged 3D pathology. Note that certain artifacts inherent to 2D histology, such as cracks, folds, and regions of poor staining quality, are eliminated and/or reduced with non-destructive 3D pathology.

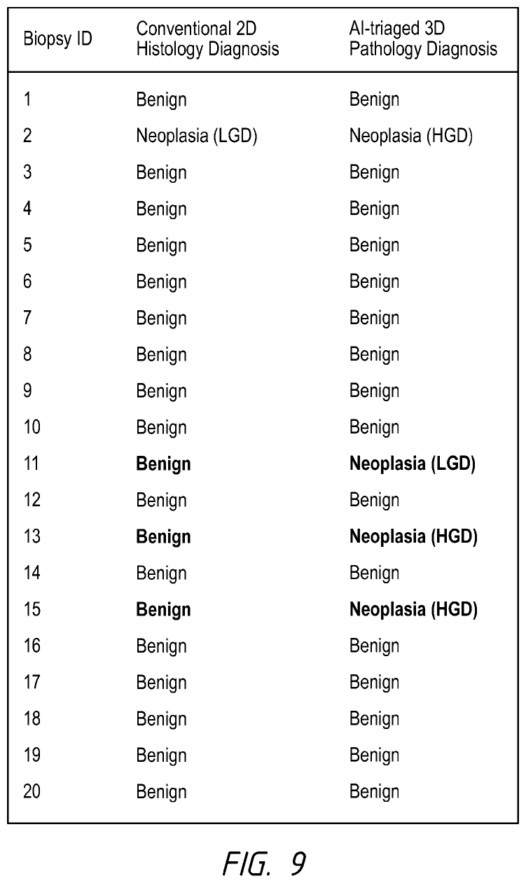

FIG. 9 shows a comparison table listing an independent validation cohort of the twenty endoscopic biopsies evaluated with conventional 2D histology and AI-triaged 3D pathology in accordance with examples described herein.

Of the twenty biopsies evaluated, three biopsies, #11, #13 and #15, diagnosed as benign by conventional histology of sixteen or less physical tissue sections were found to contain neoplasia based on AI-triaged 3D pathology of three sections as shown in the comparison table of FIG. 9.

### Discussion

The severe sampling limitations associated with conventional histology reduces the sensitivity for detecting neoplasia through regular endoscopic screening of BE patients. Non-destructive 3D pathology provides comprehensive visualization of whole biopsies, which may improve detection sensitivity and facilitate earlier intervention for patients, which is critical for maximizing their outcomes. However, it would be prohibitively time-consuming for pathologists to sift through large 3D pathology datasets. Therefore, examples described herein include an AI-based triage method to identify certain (e.g., the most critical) 2D image sections within each 3D dataset for pathologists to review. DL based triage of 3D pathology datasets may improve diagnostic sensitivity in comparison to gold-standard 2D histology while reducing pathologist workloads (e.g., the number of images that must be viewed) compared with standard histopathology practice.

As machine learning and DL based approaches continue to be developed at a rapid rate by researchers, developing methods that are interpretable will encourage clinical translation. Examples of multi-step processes described herein facilitate interpretability by providing a quantitative heat map of high-risk regions in 3D before selecting the most critical 2D image sections for pathologist review. This intermediate checkpoint provides pathologists with the opportunity to review which tissue regions and pathological structures are being ranked as having the highest probability of being neoplastic to gain confidence in the algorithm's decision-making.

Although the performance of the method enables overall improved sensitivity in comparison to conventional histology, the RFC's task is particularly challenging as it should desirably learn to recognize and correctly prioritize various manifestations of neoplasia. For example, the RFC may evaluate whether image sections with large diffuse regions of neoplasia should be ranked as more important than image sections with small focal areas of neoplasia. To facilitate learning of these distinctions, features (see Methods) are used that would account for some of these variables as inputs to the RFC. For example, the “maximum predicted probability” feature (e.g., a feature for predicted probability greater than a threshold and/or greater than other regions) enables the RFC to consider image sections with small focal areas of neoplasia, whereas the “number of patches for which P>0.10” (e.g., a feature for number of patches having probability over a threshold) feature allows the RFC to consider image sections with large regions of neoplasia. In this manner, particular feature selection for the RFC's use may enable improved selection of the most-important image sections for pathologist review.

This study was intended to facilitate the initial adoption of 3D pathology into clinical settings by designing a diagnostic workflow that is simple, familiar, and time-efficient. This motivated a choice to provide pathologists with 2D image sections, which pathologists are accustomed to viewing, rather than image stacks or videos of 3D data. It should be noted, however, that pathologist review of 3D data may enhance diagnostic accuracy, especially for structures that can be ambiguous when viewed as 2D images. Note that, in other examples, high-priority 3D image regions may be displayed as image stacks or volumetric renderings. Finally, in some examples, algorithms may classify neoplastic regions based on 3D chunks instead of 2D patches, which could improve the accuracy of DL models through the incorporation of novel 3D features.

Finally, note that in some examples, pixel-level annotations are used to train the patch-based DL algorithm. This may be undesirable in some situations. Accordingly, in some examples, weakly-supervised learning may be used to facilitate direct image-based predictions after training with image-based or patient-based labels, and may have the ability to generate attention maps that offer a similar level of interpretability to pathologists. However, it can be challenging to achieve accurate performance with such single-step methods with relatively small training datasets, as used in the particular study for which results are provided herein.

Accordingly, non-destructive 3D pathology methods may be used to enable comprehensive histologic evaluation of whole biopsies and a DL based computational triage method may be used to identify particular (e.g., the most important) 2D image sections for time-efficient pathologist review. The AI-triaged 3D pathology has the potential to improve diagnostic accuracy while reducing clinician workloads.

From the foregoing it will be appreciated that, although specific embodiments have been described herein for purposes of illustration, various modifications may be made while remaining within the scope of the claimed technology.

Examples described herein may refer to various components as “coupled” or signals as being “provided to” or “received from” certain components. It is to be understood that in some examples the components are directly coupled one to another, while in other examples the components are coupled with intervening components disposed between them. Similarly, signals or communications may be provided directly to and/or received directly from the recited components without intervening components, but also may be provided to and/or received from the certain components through intervening components.

What is claimed is:

1. A method comprising:
analyzing a three-dimensional (3D) data set of one or more medical images using an artificial intelligence (AI) model to identify two-dimensional (2D) regions of interest in the 3D data set having an increased probability of a diagnostic state; and
providing the regions of interest to another process for additional analysis.

2. The method of claim 1, wherein said analyzing the 3D data set comprises:
generating a 3D heat map, a 3D segmentation, or both, of the 3D data set using the AI model; and
identifying the regions of interest using another AI model,
wherein each region of the regions of interest comprises a respective 2D data set.

3. The method of claim 2, wherein the AI model comprises either a deep learning model or a patch-based neural network and the another AI model comprises a machine learning classifier.

4. The method of claim 3, wherein the AI model comprises at least one of convolutional neural networks (CNNs) or vision transformers (ViTs).

5. The method of claim 3, wherein the another AI model comprises at least one of support vector machines (SVM), RFCs, or k-means classifiers.

6. The method of claim 3, wherein the machine learning classifier is trained using features configured to account for a difference between smaller focal areas and larger or diffuse areas.

7. The method of claim 6, wherein the features comprise:
a ranking of predicted probability of a patch within an image;
a number of patches having probability over a threshold; and
a standard deviation of probabilities of patches in the image.

8. The method of claim 1, wherein the diagnostic state is neoplasia.

9. The method of claim 8, wherein the neoplasia is esophageal neoplasia.

10. The method of claim 8, wherein the neoplasia is prostate cancer.

11. The method of claim 1, wherein the one or more medical images comprise an image of an esophageal specimen.

12. The method of claim 1, wherein the one or more medical images comprise an image of a prostate specimen.

13. A system configured to receive a 3D data set of one or more medical images, the system comprising:
one or more processors configured to analyze the 3D data set of one or more medical images using an AI model to identify 2D regions of interest in the 3D data set having an increased probability of a diagnostic state, and further configured to provide the 2D regions of interest to another process for additional analysis.

14. The system of claim 13, wherein the one or more processors are configured to generate a 3D heat map, a 3D segmentation, or both, of the 3D data set using the AI model, and further configured to identify the regions of interest using another AI model, and
wherein each region of the regions of interest comprises a respective 2D data set.

15. The system of claim 14, wherein the AI model comprises a patch-based neural network and the another AI model comprises a machine learning classifier.

16. The system of claim 15, wherein the AI model comprises at least one of CNNs or ViTs.

17. The system of claim 15, wherein the another AI model comprises at least one of SVMs, RFCs, or k-means classifiers.

18. The system of claim 15, wherein the machine learning classifier is trained using features configured to account for a difference between smaller focal areas and larger or diffuse areas.

19. The system of claim 18, wherein the features comprise:
a ranking of predicted probability of a patch within an image;
a number of patches having probability over a threshold; and
a standard deviation of probabilities of patches in the image.

20. A non-transitory computer readable medium encoded with instructions which, when executed, cause a system to perform operations comprising:
analyzing a 3D data set of one or more medical images using an AI model to identify 2D regions of interest in the 3D data set having an increased probability of a diagnostic state; and
providing the 2D regions of interest to another process for additional analysis.

21. The non-transitory computer readable medium of claim 20, wherein said analyzing the 3D data set comprises:
generating a 3D heat map, a 3D segmentation, or both, of the 3D data set using the AI model; and
identifying the regions of interest using another AI model,
wherein each region of the regions of interest comprises a respective 2D data set.

22. The non-transitory computer readable medium of claim 21, wherein the AI model comprises at least one of CNNs or ViTs.

23. The non-transitory computer readable medium of claim 21, wherein the another AI model comprises at least one of SVMs, RFCs, or k-means classifiers.

24. The non-transitory computer readable medium of claim 21, wherein the AI model comprises either a deep learning model or a patch-based neural network and the another AI model comprises a machine learning classifier.

25. The non-transitory computer readable medium of claim 24, wherein the machine learning classifier is trained using features configured to account for a difference between smaller focal areas and larger or diffuse areas.
